(19) United States
(12) Patent Application Publication
(10) Pub. No.: US 2006/0241607 A1
(43) Pub. Date: Oct. 26, 2006

(54) Metatarsal fixation plate

(76) Inventors: Mark Myerson, Baltimore, MD (US); Priya Prasad, Warsaw, IN (US); Roy Sanders, Tampa, FL (US); Chris Bremer, Warsaw, IN (US)

Correspondence Address: MAGINOT, MOORE & BECK, LLP;CHASE TOWER, 111 MONUMENT CIRCLE, SUITE 3250, INDIANAPOLIS, IN, 46204, US

(21) Appl. No.: 11/094,954

(22) Filed: Mar. 31, 2005

(51) Int. Cl. A61F 2/30

(52) U.S. Cl. 606/69

(57) ABSTRACT

A fixation device for reduction of bone segments of the metatarsus comprises a fixation plate that is sized and configured to generally conform to the shaft of the metatarsus between the head and base. The plate includes a head end that is wider than the base end, with curved side edges that gradually taper between the two ends and produce a necked-down portion between the two ends that narrower than either end. The profile of the plate is further reduced by curving the plate across its width and along the entire length of the plate and by forming the plate at a minimal thickness of about 0.9 mm. The plate is provided with a plurality of screw holes staggered across the length and width of the plate. In the preferred embodiment, two screw holes are provided at each end and a group of four screws are provided adjacent each of the side edges. The screws holes are staggered to avoid conflict of the screws within the bone regardless of how many screws are used to secure the plate to the bone.

### BACKGROUND OF THE INVENTION

The present invention relates to a plate for use in fixation of the metatarsal bones of the foot for fracture reduction, hallux valgus correction and similar surgical procedures.

A hallux valgus deformity is a very common foot disorder, often referred to as a bunion. The most common cause attributed to this deformity is improper or ill-fitting footwear, although other biomechanical, traumatic and metabolic factors may actually be more likely etiologies for this condition. The hallux valgus deformity manifests itself as a medial deviation of the first metatarsal, and a lateral deviation and/or rotation of the hallux. This condition can lead to painful motion of the joint, difficulty in wearing shoes, and adjustments in gait that may ultimately cause problems farther up the leg. In minor cases, orthotics may alleviate, but not correct, the deformity.

The goals of surgical approaches to the treatment of a hallux valgus deformity are to relieve symptoms and pain, restore function and correct or reduce the deformity. For some indications, the surgical treatment is limited to removal of a medial portion of the metatarsus to reduce the prominence of the bunion. In more severe cases, or for elderly patients, resectional arthroplasty of the metatarso-phalangeal (MTP) joint, with or without implant, or joint arthrodesis may be indicated, although these procedures result in loss of motion at that joint.

In the majority of hallux valgus cases, the surgical treatment involves an osteotomy to correct the structural deformities associated with the cuneiform, metatarsal and phalanges. The osteotomy is often accompanied by a lateral release and capsulorraphy to address adaptive changes of the associated cartilage and ligaments. The goal is to return the metatarsus and MTP joint to their optimum anatomic positions and restore acceptable hallux valgus, inter-metatarsal angle and distal metatarsal angles. While phalanx and cuneiform osteotomies have been implemented, the prevalent procedure involves removing a wedge from the distal or head end of the metatarsus. Chevron osteotomies are typically used for correction of mild deformities, while Mitchell's transverse osteotomies are used for moderate deformities. The more severe cases usually require a Scarf osteotomy in which a Z-shaped osteotomy in the transverse plane extends from the metatarsal head to the base. Depending upon the nature of the deformity, the osteotomy may involve removal of bone (closing wedge osteotomy) or insertion of a wedge of bone (opening wedge osteotomy).

With all of these osteotomies, solid fixation of the bone segments is essential to achieve complete union. The same is true for reduction of a fracture of the metatarsus. Metatarsal fractures are relatively common conditions and range in severity from an incomplete separation, to complete separation with displacement, to severe comminution.

In the past, one typical surgical approach to correcting metatarsal fractures has been to fix the bone segments using bone screws passing from segment to segment. Other approaches include the use of cerclage wire, internal medullary pinning with K-wire, and mono-filament wire fixation. In some cases, a flat plate has been mounted to the bone bridging the fracture or osteotomy opening. These flat plates are typically limited to providing axial compression for simple fractures of the metatarsal long bone.

There is a need for a fixation device that can be used for any surgical treatment of the metatarsus, especially of the first metatarsus. The need extends further to a fixation device that accurately reduces the fracture or osteotomy and maintains the reduction until union occurs.

### SUMMARY OF THE INVENTION

To address the need for a fixation device for use in reducing the metatarsus, the present invention provides a fixation plate that is sized and configured to generally conform to the shaft of the metatarsus between the head and base. The plate includes a head end that is wider than the base end, with curved side edges that gradually taper between the two ends. In one feature of the invention, the side edges are curved to produce a necked-down portion between the two end edges that has a width less than the width of either end. This contour reduces the overall profile of the plate.

The profile of the plate is further reduced by curving the plate across its width and along the entire length of the plate. The curvature corresponds to the shape of the shaft of the metatarsus. Another feature to reduce the prominence of the plate is the minimal thickness of the plate, about 0.9 mm in the preferred embodiment.

In an important feature of the invention, the plate is provided with a plurality of screw holes staggered across the length and width of the plate. In the preferred embodiment, twelve screw holes are provided so that the surgeon may select a number, combination and location of bone screws to optimally secure the plate to the bone. Moreover, the array of screw holes provided by the plate of the present invention allows the surgeon to position the screws where they are most needed to reduce a fracture or wedge osteotomy.

The screw holes are staggered to avoid conflict of the screws within the bone. In another aspect, the screw holes include a circumferential chamfer to accommodate bone screws of different sizes.

One benefit of the fixation plate of the present invention is that it provides the surgeon with great flexibility in screw placement to reduce a fracture and maintain this reduction until union occurs. Another benefit is that the plate has a minimal prominence or profile above the bone, which reduces soft tissue trauma and minimizes the ability to visibly detect the presence of the plate in the patient's toe. These and other benefits and objects of the invention may be discerned from the following written description taken together with the accompanying figures.

### DESCRIPTION OF THE FIGURES

FIG. 1 is an enlarged view of the first ray of the foot, including the metatarsus and phalanx, with a fixation plate in accordance with one embodiment of the invention mounted thereon.

FIG. 2 is a top elevational view of the fixation plate shown in FIG. 1.

FIG. 3 is an end elevational view of the fixation plate of FIG. 2.

FIG. 4 is a side elevational view of the fixation plate of FIG. 2.

### DESCRIPTION OF THE PREFERRED EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

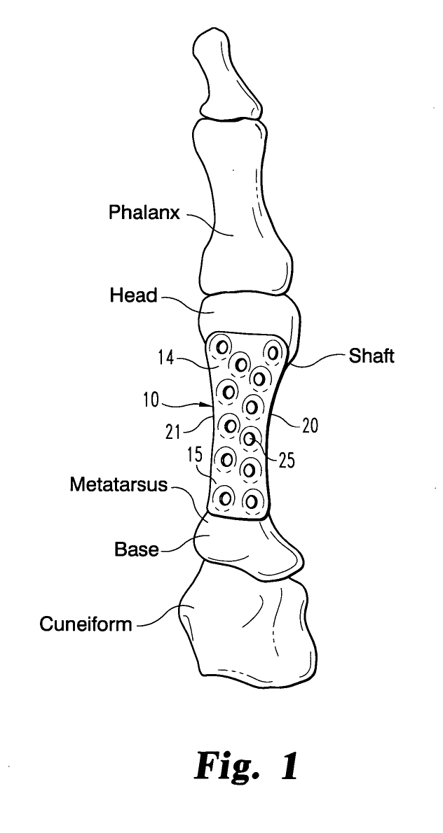

The first ray of the foot is depicted in FIG. 1 with a plate 10 mounted thereon in accordance with one embodiment of the invention. The first ray includes the cuneiform, the metatarsus and the phalanges. The plate 10 is configured for reducing fractures or bone segments of the shaft metatarsus, preferably between the base and the head of that bone.

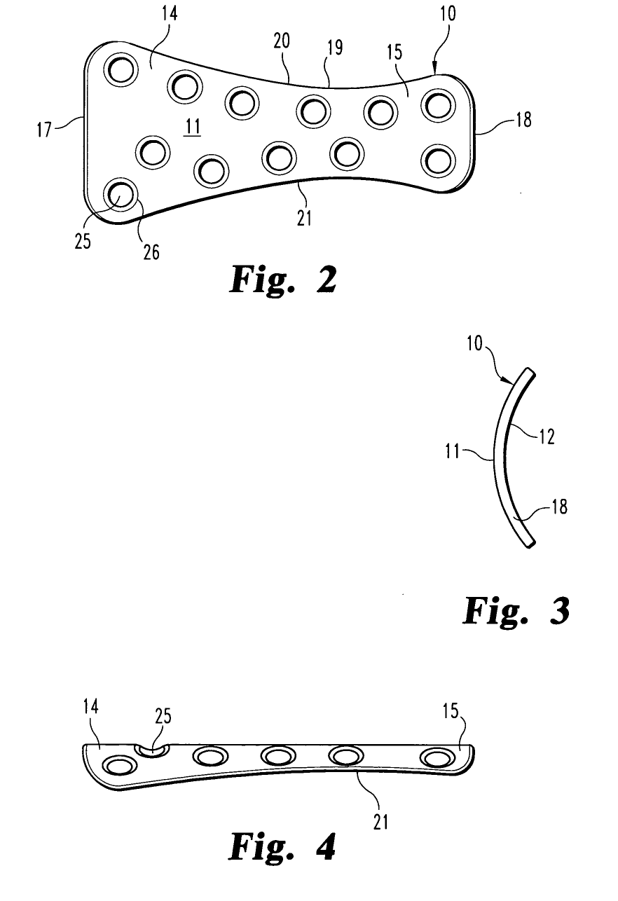

Referring to FIGS. 2-4, more specific details of the plate 10 are shown. The plate 10 includes an upper surface 11 and a lower surface 12 that is configured for substantially flush mounting onto the shaft of the metatarsus. In particular, the lower surface 12 is formed at a radius that approximates the radius of the metatarsus. In a specific embodiment, the radius is about 15.0 mm. In order to further reduce the prominence of the plate above the bone, the plate has a substantially uniform thickness between the upper and lower surfaces 11, 12 that is minimized without compromising strength of the plate. It has been found in a specific embodiment that a thickness of less than 1.0 mm, and preferably about 0.9 mm, provides a minimal profile above the bone while retaining sufficient strength to reduce and maintain the reduction for union to occur in the bone. The curvature of the plate (as shown best in FIG. 3) increases the bending stiffness of the plate. In the preferred embodiment, the plate is formed of a biocompatible metal, such as titanium. In a specific embodiment, the plate is formed of a titanium alloy, such as TI-6AL-4V.

The plate 10 includes a head end 14 that is wider than the base end 15, to generally correspond to the geometry of the metatarsus which is narrower at its base than at its head. In a preferred embodiment, the head edge 17 has a width of about 21.0 mm while the base edge 18 has a width of about 14.0 mm.

The prominence of the plate 10 between the two end edges 17, 18 is minimized by curving the side edges 20, 21. In particular, the edges are curved at a large radius so that the plate includes a necked-down region 19 that has a width less than the width of the base end edge 18. In a specific embodiment, the side edges are curved at different radii to conform more closely to the geometry of the shaft of the metatarsus. In a specific embodiment, the side edge 20, which is the medial edge in FIG. 1, is curved at a radius of about 43.0 mm. The opposite side edge 21, or the lateral edge, is curved at a radius that is more than twice the radius of the medial edge. In a specific embodiment, the lateral edge 21 is curved at a radius of about 90.0 mm. The two curved side edges result in a plate width at the necked-down region of about 12.0 mm.

As can be seen in FIG. 2, the corners of the plate between end edges 17, 18 and side edges 20, 21 are curved, preferably at a radius of about 3.8 mm. In addition, all of the edges of the plate 10 are curved across the thickness between the upper and lower surfaces 11, 12. These rounded corners and edges reduce the trauma to soft tissue surrounding the metatarsus.

In accordance with one feature of the invention, the plate 10 defines a plurality of holes 25 therethrough that are configured to receive a bone engaging fastener, such as a bone screw. The holes may be configured to receive a non-locking or a self-locking screw. In the latter instance, the holes 25 may be tapered at a conventional angle (such as 12°) that is acceptable for a locking screw.

In the preferred embodiment, each hole 25 includes a circumferential chamfer 26 that allows the hole to accommodate differently sized screws. In a specific embodiment, the holes 25 may accept 2.7 mm or 3.5 mm locking or non-locking cortical screws to secure the plate 10 to the bone.

In a further feature of the plate 10, the screw holes 25 are staggered throughout the entire width and length of the plate. This pattern of screw holes provides significant advantages. One advantage is that the plate can accommodate several screw holes to increase the versatility of the plate. In the preferred embodiment, a plate 10 for the first metatarsal includes twelve screw holes from which the surgeon may select. If the deformity being treated is a fracture of the metatarsus, the surgeon may select among several combinations of two or more screws on either side of the fracture. If the metatarsus includes multiple fractures, the multiple screw holes virtually ensure that every bone segment can be reduced and fixed until union occurs. If a wedge osteotomy at the head of the metatarsus is being reduced, the screws may be placed at the pair of screw holes at the base end 15 of the plate and any number of screw positions may be selected at the head end 14 depending upon the position and type of osteotomy.

Another advantage of the arrangement of the screw holes 25 on plate 10 is that no two screws will conflict within the metatarsus bone, even if a bone screw is driven through every screw hole 25. While the curvature of the plate produces the benefits discussed above, it creates the risk of conflict between adjacent screws. Staggering the screw holes as shown in FIG. 2 eliminates the possibility of such conflicts. In a preferred embodiment of the invention, the pairs of screws at the head end 14 and base end 15 may be transversely aligned because the pairs of holes are separated by a sufficient width to avoid contact within the bone. Of the remaining screws along the interior length of the plate 10 the screw holes adjacent the medial edge 20 are preferably staggered by about 3.0 mm relative to the screw holes adjacent to the lateral edge 21.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

What is claimed is:

1. A fixation device for reduction of bone segments of the metatarsus comprising:
an elongated plate having a head end and a base end, said plate having a width at said head end that is greater than the width at said base end;
said plate further including opposite curved side edges between said head and base ends, said side edges curved to produce a necked-down portion of said plate between said head and base ends, wherein said necked-down portion has a width less than the width of said base end; and
said plate further defining a pair of holes at each of said head end and said base end and a plurality of holes between said head end and said base end.

2. The fixation device of claim 1, wherein said plurality of holes are staggered relative to each other.

3. The fixation device of claim 2, wherein said plurality of holes include a first group of holes adjacent one of said side edges and a second group of holes adjacent the opposite one of said side edges and staggered relative to said first group of holes.

4. The fixation device of claim 3, wherein said first and second groups of holes each includes at least two holes.

5. The fixation device of claim 1, wherein said elongated plate is curved between said side edges substantially uniformly along the length of said plate.

6. The fixation device of claim 1, wherein at least some of said holes includes a circumferential chamfer configured so that said hole accepts differently sized screws therethrough.

7. The fixation device of claim 1, wherein said plate has a thickness that is less than about one (1) mm.

8. The fixation device of claim 1, wherein one of said side edges is curved at a first radius and the opposite one of said side edges is curved at a second radius greater than said first radius.
